(19) United States
(12) Patent Application Publication
(10) Pub. No.: US 2015/0320985 A1
(43) Pub. Date: Nov. 12, 2015

(54) BALLOON CATHETER

(71) Applicant: ASAHI INTECC CO., LTD., Nagoya-shi (JP)

(72) Inventor: Yuta NAKAGAWA, Osaka-shi (JP)

(21) Appl. No.: 14/620,505

(22) Filed: Feb. 12, 2015

(30) Foreign Application Priority Data
May 10, 2014 (JP) 2014-098193

(51) Int. Cl. A61M 25/10

(52) U.S. Cl. CPC A61M 25/1006; A61M 25/1002; A61M 2025/1004

(57) ABSTRACT

A balloon catheter including an inner tube and a balloon secured to the inner tube, in which a rough portion that has a large surface roughness and a smooth portion that has a surface roughness that is smaller than the large surface roughness of the rough portion are formed on a surface of the balloon in a circumferential direction. When the balloon is folded on an outer periphery of the inner tube, the rough portion is positioned between the inner tube and the smooth portion.

### CROSS REFERENCE TO RELATED APPLICATION

This application claims priority to Japanese Patent Application No. 2014-098193 filed in the Japan Patent Office on May 10, 2014, the entire contents of which are incorporated herein by reference.

### BACKGROUND

The disclosed embodiments relate to a medical device. Specifically, the disclosed embodiments relate to a balloon catheter to be inserted into a stenosis site or an occlusion site formed in a tubular organ such as a blood vessel, a bile duct, a pancreatic duct, or the like, and that dilates the stenosis site or the occlusion site to secure the flow of blood, bile (biliary fluid), pancreatic juice, or the like.

When a stenosis site or an occlusion site is formed in a tubular organ (e.g., a blood vessel, a bile duct, a pancreatic duct, or the like), the flow of fluid through the organ becomes restricted. Balloon catheters have been widely used to treat a stenosis site or an occlusion site.

A balloon catheter mainly includes a balloon as a dilation body, an outer tube that is secured to the proximal end of the balloon, and an inner tube that is inserted inside the balloon and the outer tube. The inner tube is used to insert a guide wire, and a dilation lumen provided between the outer tube and the inner tube is used for communicating liquid (e.g. a contrast medium or a saline solution) for dilating the balloon.

When a balloon catheter is inserted into a tubular organ and frictional resistance between the balloon catheter and the tubular organ is large, the balloon catheter can become caught before reaching the stenosis site or the occlusion site. In order to address the above problem, a coating agent may be coated onto the balloon catheter to reduce the frictional resistance between the balloon catheter and the tubular organ (see Japanese Patent No. 5330506, for example).

In the balloon catheter of Japanese Patent No. 5330506, a composition having a low concentration of coating agent and a composition having a high concentration of coating agent are coated in multi-layers so as to reduce frictional resistance between the balloon catheter and the tubular organ and so as to maintain manipulability of the balloon catheter even when some of the coating agent comes off during a procedure.

However, in the balloon catheter of Japanese Patent No. 5330506, since the coating agent is coated on the entire surface of the balloon, the balloon disadvantageously slips in the proximal-distal direction when the balloon is dilated in the stenosis site or the occlusion site. Furthermore, although the coating agent is coated in multi-layers, some of the coating agent still comes off during the procedure. In particular, when the stenosis site or the occlusion site is formed at the end of the tubular organ and the balloon catheter must traverse a large distance, the coating agent comes off and the manipulability of the balloon catheter disadvantageously decreases. Furthermore, since the coating agent is coated in multi-layers, the process of manufacturing the balloon catheter is lengthy and, as a result, the cost to manufacture the balloon catheter is disadvantageously high.

### SUMMARY

The disclosed embodiments have been devised to address the above circumstances. In particular, a balloon catheter of the disclosed embodiments includes a balloon having, on an outer surface, a rough portion that has a large surface roughness and a smooth portion that has a small surface roughness (compared to the large surface roughness of the rough portion). By positioning the rough portion between an inner tube of the balloon catheter and the smooth portion when the balloon is folded, frictional resistance between the balloon catheter and the tubular organ is reduced without requiring a coating agent, and the balloon does not easily slip in the proximal-distal direction when the balloon is dilated.

A balloon catheter of the disclosed embodiments includes an inner tube and a balloon secured to the inner tube. A rough portion that has a large surface roughness and a smooth portion that has a surface roughness that is smaller than that of the rough portion are formed on a surface of the balloon in a circumferential direction, and when the balloon is folded on an outer periphery of the inner tube, the rough portion is positioned between the inner tube and the smooth portion. That is, only the smooth portion is exposed.

When a technician inserts the balloon catheter into a tubular organ, only the smooth portion of the balloon comes in contact with the wall of the tubular organ. Thus, frictional resistance between the balloon catheter and the tubular organ is reduced and, as a result, the manipulability of the balloon catheter is improved. Furthermore, when the technician dilates the balloon in the stenosis site or the occlusion site, the rough portion of the balloon comes in contact with the wall of the tubular organ. The frictional resistance between the balloon catheter and the wall of the tubular organ is therefore increased and, as a result, the risk of the balloon slipping in the proximal-distal direction is reduced. Moreover, since no coating agent is required, the manipulability of the balloon catheter is maintained even if the balloon catheter must traverse a large distance because there is no risk of increased resistance due to a coating agent coming off of the balloon.

### BRIEF DESCRIPTION OF THE DRAWINGS

FIG. 1 is a general view of a balloon catheter (dilated) of the disclosed embodiments.

FIG. 2 is a diagram illustrating a cross-section taken along line II-II of FIG. 1.

FIG. 3 is another general view of the balloon catheter of FIG. 1 (folded).

FIG. 4 is a diagram illustrating a cross-section taken along line IV-IV of FIG. 3.

FIG. 5 is a diagram illustrating a cross-section of another balloon catheter (dilated) of the disclosed embodiments.

FIG. 6 is a diagram illustrating a cross-section of the balloon illustrated in FIG. 5 in a folded state.

FIG. 7 is a diagram illustrating a cross-section of another balloon catheter (folded) of the disclosed embodiments.

FIG. 8 is a diagram illustrating a cross-section of another balloon catheter (dilated) of the disclosed embodiments.

FIG. 9 is a diagram illustrating a cross-section of the balloon illustrated in FIG. 8 in a folded state.

### DETAILED DESCRIPTION OF EMBODIMENTS

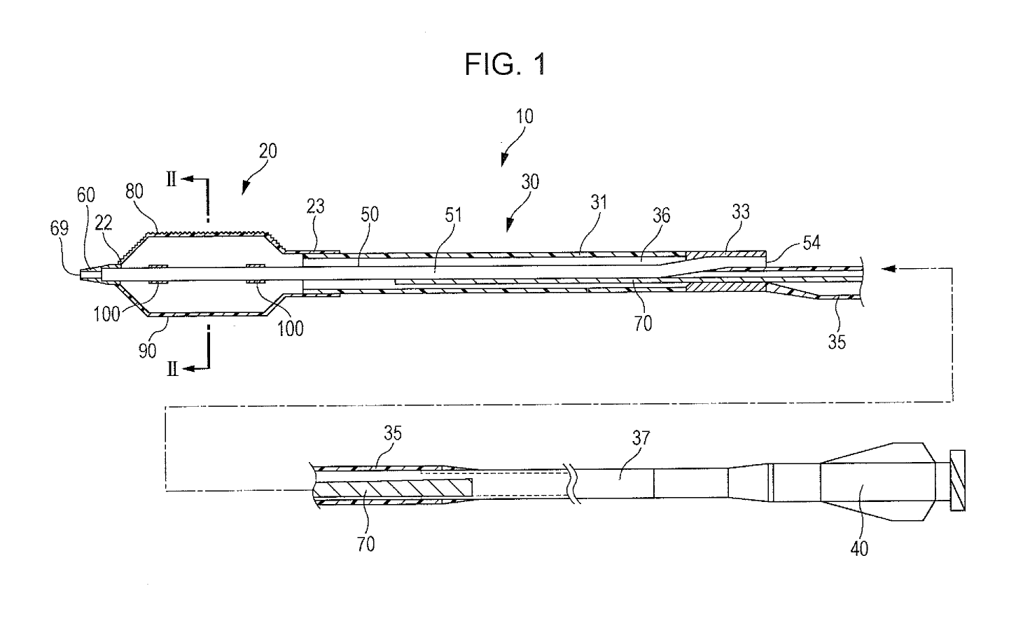

Referring to FIGS. 1 to 4, a balloon catheter 10 of the disclosed embodiments will be described. In FIGS. 1 to 3, the left side of the drawing corresponds to a distal end of the balloon catheter 10 that is to be inserted into the body, and the right side of the drawing corresponds to a proximal end of the balloon catheter 10 that is to be manipulated by a technician, such as a doctor.

The balloon catheter 10 may be used to treat, for example, a stenosis site or an occlusion site that is formed in a blood vessel, a bile duct, a pancreatic duct, or the like. As illustrated in FIG. 1, the balloon catheter 10 mainly includes a balloon 20, an outer tube 30, a connector 40, an inner tube 50, a tip 60, and a reinforcement body 70. Note that FIG. 1 illustrates a dilated state of the balloon 20.

The balloon 20 that dilates a stenosis site or an occlusion site is a member formed of resin and includes a distal end attaching portion 22 on the distal end and a proximal end attaching portion 23 on the proximal end. The distal end attaching portion 22 is secured to the distal end of the inner tube 50 and to the tip 60, and the proximal end attaching portion 23 is secured to the distal end of the outer tube 30. In FIG. 1, although the distal end attaching portion 22 is secured to the distal end of the inner tube 50 through the tip 60, the arrangement of the distal end attaching portion 22 is not limited to the above and the distal end attaching portion 22 may be interposed between the distal end of the inner tube 50 and the tip 60. Furthermore, in FIG. 1, although the proximal end attaching portion 23 is secured to an outer periphery of the distal end of the outer tube 30, the arrangement of the proximal end attaching portion 23 is not limited to the above and the proximal end attaching portion 23 may be secured to an inner periphery of the distal end of the outer tube 30.

The outer tube 30 is a tubular member having a dilation lumen 36 for supplying liquid, such as a contrast medium or a saline solution, to dilate the balloon 20. In order from the distal end to the proximal end, the outer tube 30 includes a distal end outer tube portion 31, a guide wire port portion 33, an intermediate outer tube portion 35, and a proximal end outer tube portion 37. The distal end outer tube portion 31 and the intermediate outer tube portion 35 are tubes formed of resin, such as a polyamide, polyamide elastomer, polyolefin, polyester, or polyester elastomer. The guide wire port portion 33 is a portion where the distal end outer tube portion 31, the intermediate outer tube portion 35, and the inner tube 50 are secured.

The inner tube 50 is inserted into the distal end outer tube portion 31, and the dilation lumen 36 described above is formed between the distal end outer tube portion 31 and the inner tube 50.

The proximal end outer tube portion 37 is a metal tubular member that is a so-called hypo tube. The distal end of the proximal end outer tube portion 37 is inserted into and is secured to the proximal end of the intermediate outer tube portion 35. The connector 40 is attached to the proximal end of the proximal end outer tube portion 37. When liquid, such as a contrast medium or a saline solution, for dilating the balloon 20 is supplied from an indeflator (not shown) that is attachable to the connector 40, the liquid passes through the dilation lumen 36 and dilates the balloon 20. Note that the material of the proximal end outer tube portion 37 is not limited to a particular material and stainless steel (SUS304) or a super-elastic alloy, such as a Ni—Ti alloy, may be used.

The inner tube 50 forms a guide wire lumen 51 for inserting a guide wire therein. Furthermore, the proximal end of the inner tube 50 is secured to the guide wire port portion 33 of the outer tube 30 so as to form a proximal end guide wire port 54.

The distal end of the inner tube 50 is secured to the tip 60 and the distal end attaching portion 22 of the balloon 20. The tip 60 is a cylindrical member having a tapered outer shape whose outside diameter gradually decreases towards the distal end, and is formed of flexible resin. The resin forming the tip 60 is not limited to a particular resin and, for example, a polyurethane or polyurethane elastomer may be employed. The tip 60 is secured to the distal end of the guide wire lumen 51 and includes a distal end guide wire port 69 on the distal end thereof.

Two radiopaque markers 100 are attached to the inner tube 50 inside the balloon 20 so as to enable the position of the balloon 20 to be perceived when under radiation exposure.

The reinforcement body 70 is attached to an inner circumferential surface of the distal end of the proximal end outer tube portion 37. The reinforcement body 70 has a round cross-section and is a metal wire rod having a tapered shape whose diameter decreases towards the distal end. The material of the reinforcement body 70 is not limited to a particular material and stainless steel (SUS304) or a supra-elastic alloy, such as a Ni—Ti alloy, may be used.

The reinforcement body 70 passes through the intermediate outer tube portion 35 and the guide wire port portion 33 and extends to the distal end outer tube portion 31. In FIG. 1, although the distal end of the reinforcement body 70 is not in contact with both the outer tube 30 and the inner tube 50, the arrangement is not limited to the above arrangement. For example, the distal end of the reinforcement body 70 may be interposed between the outer tube 30 and the inner tube 50 so as to be in contact with both the outer tube 30 and the inner tube 50.

A rough portion 80 that has a large surface roughness and a smooth portion 90 that has a surface roughness that is smaller than the surface roughness of the rough portion 80 are formed on the surface of the balloon 20. In the rough portion 80, the heights between the protruded portions and the recessed portions are large on average, and in the smooth portion 90, the heights between the protruded portions and the recessed portions are small on average (that is, smaller than the heights between the protruded portions and the recessed portions of the rough portion 80).

The method of forming the rough portion 80 and the smooth portion 90 on the surface of the balloon 20 is not limited to a particular method. For example, after fabricating a balloon 20 having only a smooth surface, a plasma treatment may be applied to the entire balloon 20 that has been partially masked so that the masked portion becomes the smooth portion 90 and the unmasked portion becomes the rough portion 80. Alternatively, after fabricating the balloon 20 having only a smooth surface, some portions may be physically scraped off with paper or the like such that the portion that has not been scraped becomes the smooth portion 90, and the physically scraped portion becomes the rough portion 80. Alternatively, after pre-fabricating a balloon 20 having only a rough surface, some portion of the balloon 20 may be pressed with a roller that is coated with resin so that the pressed portion becomes the smooth portion 90 and the non-pressed portion becomes the rough portion 80.

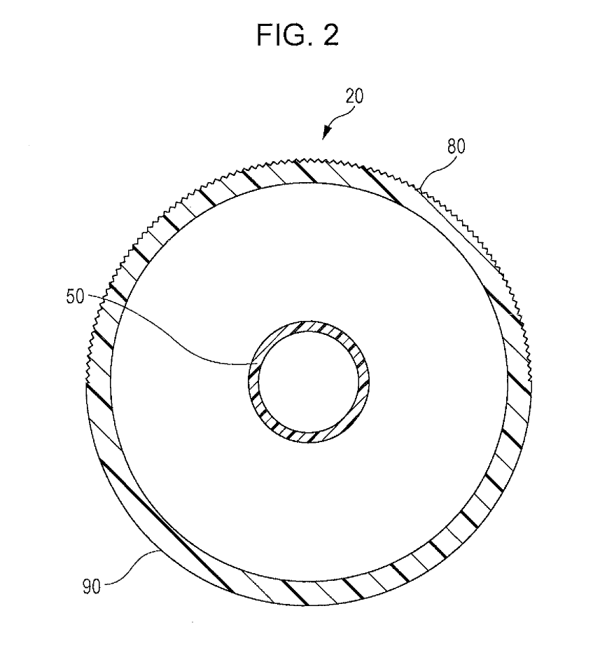

FIG. 2 illustrates a cross-section taken along line II-II of FIG. 1. As illustrated in FIG. 2, a single rough portion 80 that has a large surface roughness and a single smooth portion 90 that has a surface roughness that is smaller than the rough portion 80 are formed on the surface of the balloon 20 in a circumferential direction. That is, the rough portion 80 extends along a portion of the circumference of the balloon 20 from the proximal end to the distal end of the balloon 20, and the smooth portion 90 extends along the remaining circumference of the balloon 20 from the proximal end to the distal end of the balloon 20.

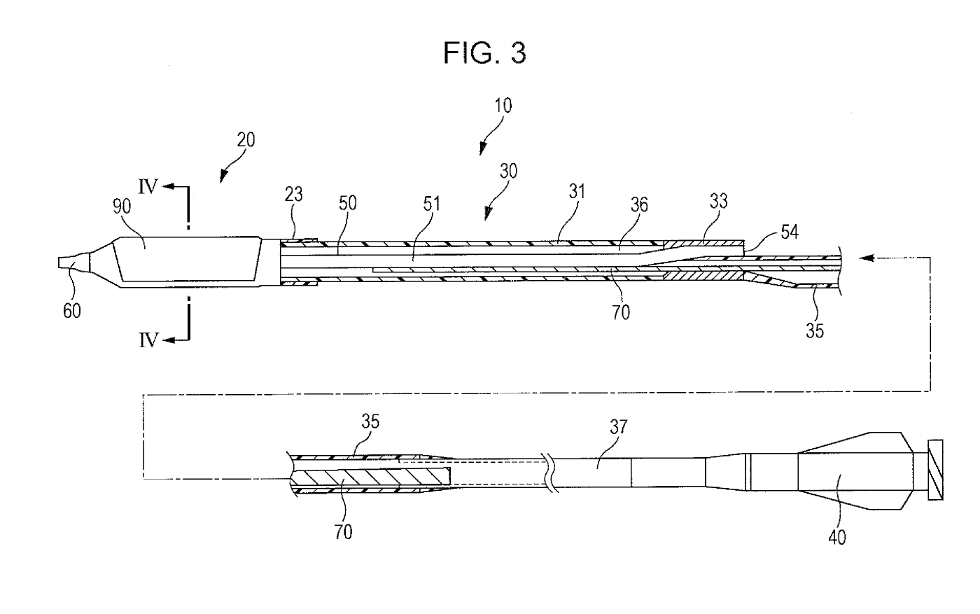

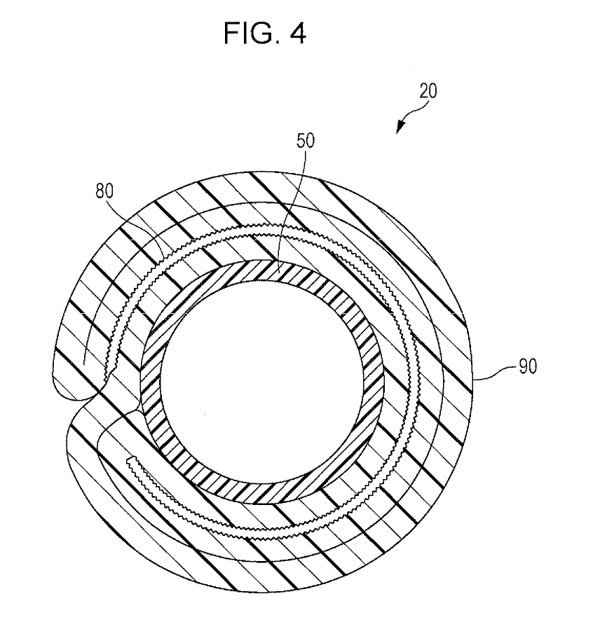

FIG. 3 illustrates a state in which the balloon 20 is folded on the outer periphery of the inner tube 50. In FIG. 3, the tip 60 and the balloon 20 are illustrated in external view rather than in cross-sectional view for convenience of description. FIG. 4 illustrates a cross-section taken along line IV-IV of FIG. 3. As illustrated in FIG. 4, when the balloon 20 is folded on the outer periphery of the inner tube 50, the rough portion 80 is positioned between the inner tube 50 and the smooth portion 90.

When the technician inserts the balloon catheter 10 into a tubular organ (e.g., a blood vessel, a bile duct, a pancreatic duct, or the like) as illustrated in FIG. 4, only the smooth portion 90 of the balloon 20 comes in contact with the wall of the tubular organ. Because the rough portion 80 of the balloon 20 is covered by the smooth portion 90, frictional resistance between the balloon catheter 10 and the tubular organ is reduced and, as a result, the manipulability of the balloon catheter 10 is improved. Furthermore, when the technician dilates the balloon 20 in the stenosis site or the occlusion site, as illustrated in FIG. 2, both the smooth portion 90 and the rough portion 80 of the balloon 20 come in contact with the wall of the tubular organ. Accordingly, the frictional resistance between the balloon catheter 10 and the wall of the tubular organ increases and, as a result, the risk of the balloon 20 slipping in the proximal-distal direction is reduced. Moreover, since there is no need to coat a coating agent on the balloon 20, manipulability of the balloon catheter 10 is maintained even if the balloon catheter must traverse a large distance because there is no risk of increased resistance due to a coating agent coming off of the balloon 20.

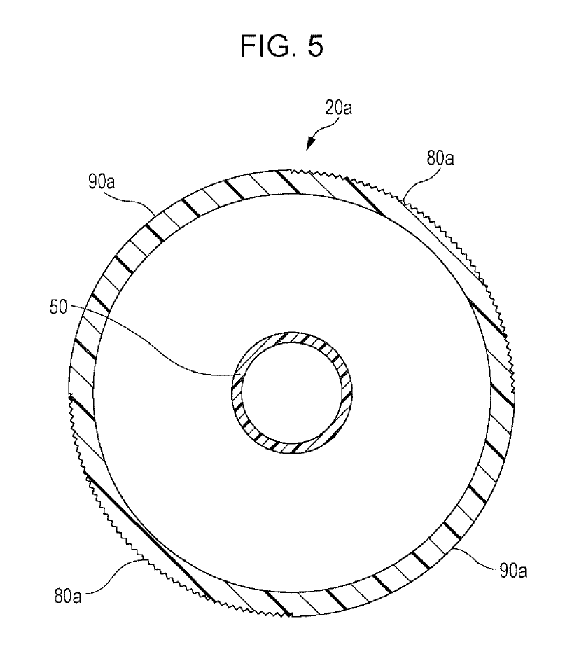

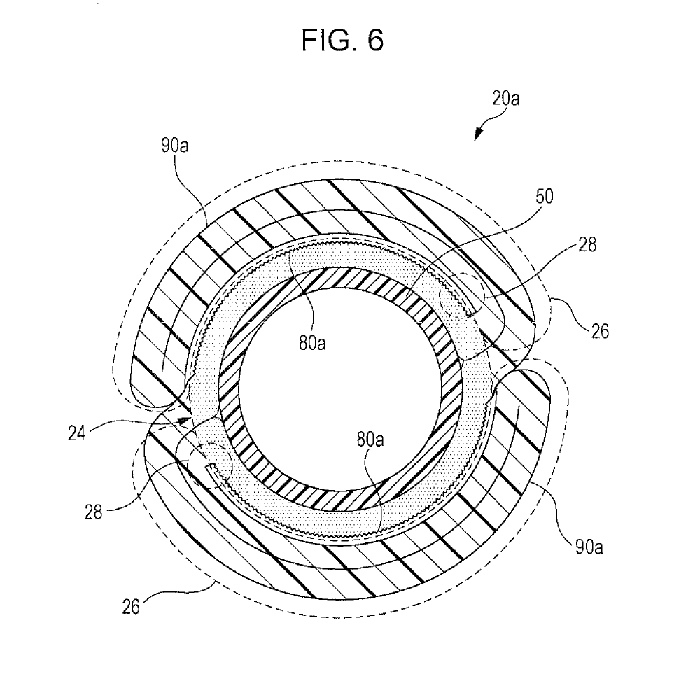

In FIG. 5, rough portions 80a that have a large surface roughness and smooth portions 90a that have a surface roughness that is smaller than that of the rough portions 80a are each formed alternately on a surface of a balloon 20a in the circumferential direction. As illustrated in FIG. 6, the balloon 20a folded on the outer periphery of the inner tube 50 includes a trunk portion 24 that covers the outer periphery of the inner tube 50, two wing portions 26 that are folded in a fixed direction so as to cover the outer periphery of the trunk portion 24, and two valley portions 28 formed at the inside bases of the wing portions 26 when the wing portions 26 are folded over the outer periphery of the trunk portion 24. Since the rough portions 80a are formed only on the outer periphery of the trunk portion 24, when the balloon 20a is folded on the outer periphery of the inner tube 50, the rough portions 80a are positioned between the inner tube 50 and the smooth portions 90a and are covered by the wing portions 26 that are formed by the smooth portions 90a.

When the technician dilates the balloon 20a in the stenosis site or the occlusion site, the plurality of rough portions 80a of the balloon 20a can come into contact with the wall of the tubular organ. Since the rough portions 80a of the balloon 20a come into contact with both the upper and lower portions of the wall of the tubular organ, the risk of the balloon 20a slipping in the proximal-distal direction can be further reduced.

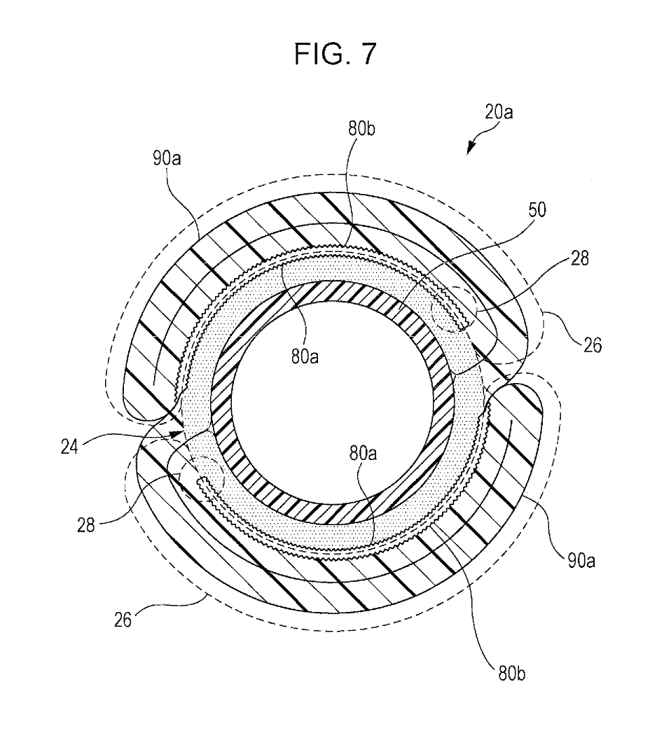

Note that in FIG. 6, although the rough portions 80a are only formed on the outer periphery of the trunk portion 24, the arrangement is not limited to the above. As illustrated in FIG. 7, rough portions 80a are formed on the outer periphery of the trunk portion 24, and rough portions 80b are formed on the surfaces of the wing portions 26 on the inner tube 50 side.

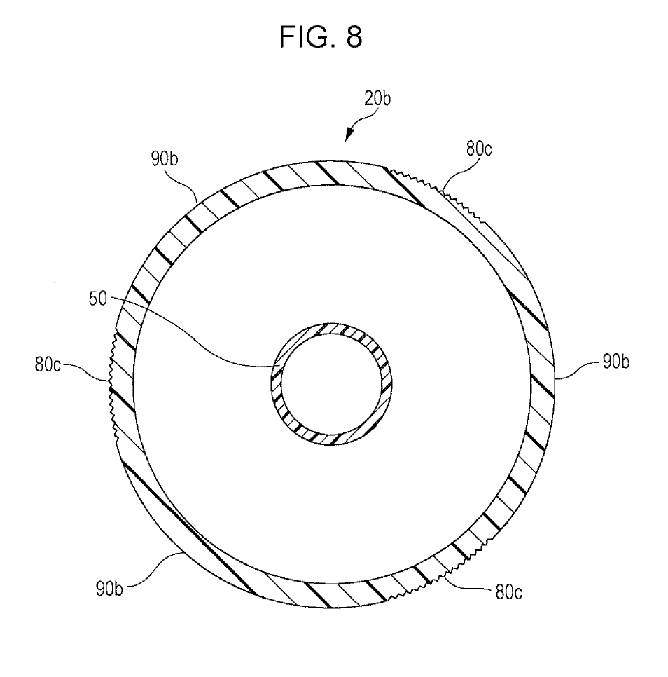

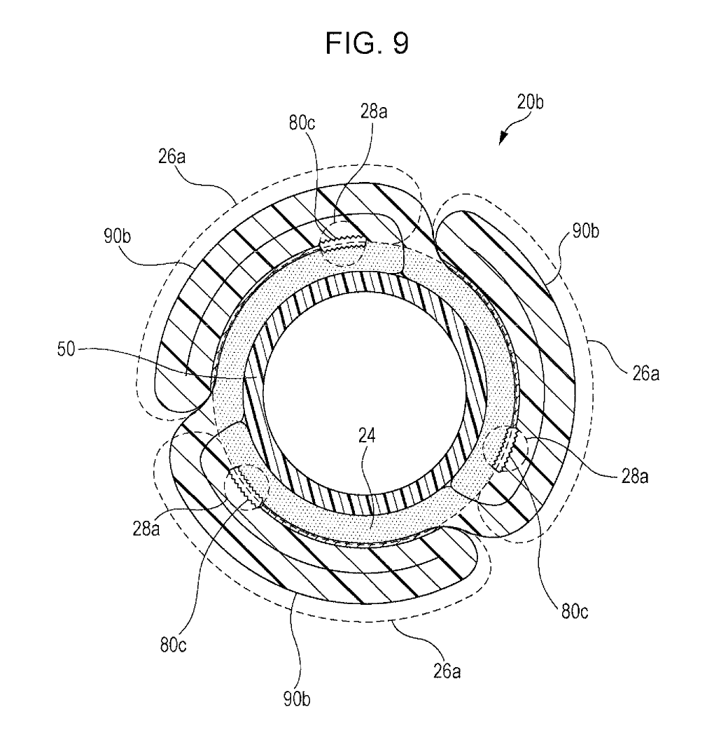

In FIG. 8, three rough portions 80c that have a large surface roughness and three smooth portions 90b that have a surface roughness that is smaller than the rough portions 80c are each formed alternately on a surface of a balloon 20b in the circumferential direction. As illustrated in FIG. 9, the balloon 20b folded on the outer periphery of the inner tube 50 includes a trunk portion 24 that covers the outer periphery of the inner tube 50, three wing portions 26a that are folded in a fixed direction so as to cover the outer periphery of the trunk portion 24, and three valley portions 28a formed at the inside bases of the wing portions 26a when the wing portions 26a are folded over the outer periphery of the trunk portion 24. The three wing portions 26a are formed by the smooth portions 90b and the three valley portions 28a are formed by the rough portions 80c. Accordingly, when the balloon 20b is folded on the outer periphery of the inner tube 50, the plurality of valley portions 28a that are formed by the rough portions 80c can be easily positioned between the inner tube 50 and the smooth portions 90b.

Furthermore, the balloon 20b may be formed by being heated and compressed inside a mold so that a portion of the balloon 20b where a contact pressure with the mold is high becomes a rough portion having a large surface roughness, and a portion of the balloon 20b where a contact pressure with the mold is low becomes a smooth portion having a small surface roughness. Thus, by heating and compressing the balloon 20b inside the mold, the three valley portions 28a are formed as the rough portions 80c where the contact pressure with the mold is high, and the three wing portions 26a are formed as the smooth portions 90b where the contact pressure with the mold is low. Accordingly, the rough portions 80c and the smooth portions 90b of the balloon 20b can be formed without requiring any additional surface treatment processes.

Note that in order to further reduce frictional resistance between the balloon catheter 10 and the wall of the tubular organ, a coating agent may be coated on the outer periphery of the smooth portions 90, 90a, and 90b of the balloons 20, 20a, and 20b. However, it is desirable that no coating agent is coated on the outer periphery of the rough portions 80, 80a, 80b, and 80c of the balloons 20, 20a, and 20b.

As described above, when the technician inserts the balloon catheter 10 into the tubular organ, only the smooth portion 90, 90a, or 90b of the balloon 20, 20a, or 20b comes in contact with the wall of the tubular organ, and frictional resistance between the balloon catheter 10 and the wall of the tubular organ is reduced. Additionally, when the technician dilates the balloon 20 inside the tubular organ, the rough portions 80, 80a, 80b, or 80c of the balloon 20, 20a, or 20b come in contact with the wall of the tubular organ, and frictional resistance between the balloon catheter 10 and the wall of the tubular organ is increased. Furthermore, since there is no need to coat a coating agent on the balloon 20, 20a, and 20b, manipulability of the balloon catheter 10 is maintained even if the balloon catheter must traverse a large distance because there is no risk of increased resistance due to a coating agent coming off of the balloon 20, 20a, and 20b.

What is claimed is:

1. A balloon catheter comprising:
an inner tube; and
a balloon secured to the inner tube,
wherein:
a rough portion that has a large surface roughness and a smooth portion that has a surface roughness that is smaller than the large surface roughness of the rough portion are formed on a surface of the balloon in a circumferential direction, and
when the balloon is folded on an outer periphery of the inner tube, the rough portion is positioned between the inner tube and the smooth portion.

2. The balloon catheter according to claim 1, wherein:
when the balloon is folded on the outer periphery of the inner tube, the balloon includes a trunk portion, a wing portion, and a valley portion, and
the rough portion is formed on the valley portion, an outer periphery of the trunk portion, and an inner periphery of the wing portion.

3. The balloon catheter according to claim 1, wherein the balloon includes a plurality of rough portions and a plurality of smooth portions.

4. The balloon catheter according to claim 3, wherein the rough portions and the smooth portions are formed alternately in the circumferential direction.

5. The balloon catheter according to claim 3, wherein:
when the balloon is folded on the outer periphery of the inner tube, the balloon includes a plurality of wing portions and a plurality of valley portions,
the smooth portions are formed on the plurality of wing portions, and
the rough portions are formed on the plurality of valley portions.

6. The balloon catheter according to claim 4, wherein
when the balloon is folded on the outer periphery of the inner tube, the balloon includes a plurality of wing portions and a plurality of valley portions,
the smooth portions are formed on the plurality of wing portions, and
the rough portions are formed on the plurality of valley portions.

7. The balloon catheter according to claim 3, wherein:
when the balloon is folded on the outer periphery of the inner tube, the balloon includes a trunk portion, a plurality of wing portions, and a plurality of valley portions,
the rough portions are formed on an outer periphery of the trunk portion, and
the smooth portions are formed on an outer periphery of the wing portions.

8. The balloon catheter according to claim 4, wherein:
when the balloon is folded on the outer periphery of the inner tube, the balloon includes a trunk portion, a plurality of wing portions, and a plurality of valley portions,
the rough portions are formed on an outer periphery of the trunk portion, and
the smooth portions are formed on an outer periphery of the wing portions.

9. The balloon catheter according to claim 7, wherein the rough portions are formed on the outer periphery of the trunk portion and an inner periphery of the wing portions.

10. The balloon catheter according to claim 8, wherein the rough portions are formed on the outer periphery of the trunk portion and an inner periphery of the wing portions.

11. The balloon catheter according to claim 1, wherein when the balloon is folded on the outer periphery of the inner tube, the rough portion is covered by the smooth portion.
